(19) United States
(12) Patent Application Publication
(10) Pub. No.: US 2014/0052170 A1
(43) Pub. Date: Feb. 20, 2014

(54) EMBOLISM PROTECTION DEVICE

(71) Applicants: Richard R. Heuser, Phoenix, AZ (US); Furqan Tejani, Yonkers, NY (US)

(72) Inventors: Richard R. Heuser, Phoenix, AZ (US); Furqan Tejani, Yonkers, NY (US)

(21) Appl. No.: 13/670,042

(22) Filed: Nov. 6, 2012

Related U.S. Application Data
(60) Provisional application No. 61/684,657, filed on Aug. 17, 2012.

(51) Int. Cl. A61F 2/01

(52) U.S. Cl. 606/200

(57) ABSTRACT

Disclosed herein are systems and methods for protecting a subject from embolisms during TAVI percutaneous valve procedures, coronary bypass surgery and heart valve surgery. Various embodiments include an intravascular embolism protection device that includes an elongated, compliant frame made from a material having a shape-memory function, and a mesh material coupled to the elongated, compliant frame, the mesh material having a pore size selected to allow blood to pass therethrough while retaining potential emboli. In some embodiments, the embolism protection device has a collapsed state wherein device is configured to fit within the lumen of a support catheter and an expanded state wherein the embolism protection device is configured to cover a plurality of arterial branch ostias.

### CROSS REFERENCE TO RELATED APPLICATIONS

The present application claims priority to U.S. Provisional Patent Application No. 61/684,657, filed Aug. 17, 2012, entitled “EMBOLISM PROTECTION DEVICE,” the disclosure of which is hereby incorporated by reference in its entirety.

### TECHNICAL FIELD

Embodiments relate to an embolism-preventing device that prevents the free flow of embolism-creating particles that are created during cardiac procedures such as percutaneous valve interventions, particularly transcatheter aortic valve implantation (TAVI) procedures, as well as coronary bypass surgery and heart valve surgery.

### BACKGROUND

An embolus can be any particle comprising a foreign or native material that enters the vascular system with potential to cause occlusion of blood flow. Emboli can be formed from aggregated fibrin, red blood cells, collagen, cholesterol, plaque, fat, calcified plaque, bubbles, arterial tissue, and/or other miscellaneous fragments. Each dislodged fragment, or embolus, is carried along by the blood flow until it becomes lodged or trapped in a smaller vessel and occludes blood flow, creating an embolism. Since emboli reduce or cut off blood flow, damage to the body may result, such as tissue damage, heart attack, stroke, or even death.

Percutaneous valve interventions include valvuloplasty, annuloplasty, and valve replacement surgeries performed on the mitral, tricuspid, aortic, and pulmonary valves. These interventions carry a high risk of embolism formation. For instance, aortic valve applications, such as transcatheter aortic valve implantation (TAVI) procedures, may carry a 60-80% chance of embolism formation and subsequent cerebral ischemic events.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an example of an embolism protection device that has been positioned over a guidewire and inside a support catheter in a desired artery;

FIG. 2 illustrates the device shown in FIG. 1 after the support catheter has been retracted to expose an elongated embolism prevention device;

FIG. 3 is a close-up view of the embolism protection device shown in FIG. 2; and

FIG. 4 illustrates an example of an embolism protection device having stabilization wires, all in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide embolism protection devices that may be deployed in a body vessel for the collection of loosened or floating debris, such as embolic material dislodged during or thrombi formed as a result of percutaneous cardiac procedure, such as a valve intervention. In some embodiments, the device may be advanced through the vasculature using a right radial artery/right brachial artery approach, and it may be positioned within the left subclavian artery in such a way that it covers all of the ostia of the major branches of the left subclavian artery. Thus, the device may protect the subject from brain embolism associated with, for instance, percutaneous treatment or replacement of the aortic valve, or any coronary stent or bypass procedure, and may be particularly useful for TAVI procedures. In other embodiments, the device may be used to protect the vasculature of a patient from dislodged materials (e.g., potential emboli and thrombi) from valvular or coronary artery disease, or during angioplasty, atherectomy, thrombectomy, embolectomy, intravascular diagnostic procedures, stent placement procedures, and/or any minimally invasive heart intervention. Embodiments of the device also may be used to protect a subject from emboli and thrombi resulting from open interventional procedures, including transapical approaches to treat valvular disease and cardiovascular bypass, as well as standard open coronary bypass and heart valve surgeries.

In various embodiments, the embolism protection device may include a compliant, elongated frame, which in some embodiments may be formed from a material having a shape memory function, such as Nitinol™. The frame may be coupled to and configured to stabilize a mesh material, which also may be formed from a material having a shape memory function, such as Nitinol™. In various embodiments, the embolism protection device may be configured to be advanced in a collapsed state along a guidewire to a desired position within an artery, such as the left subclavian artery, where it may be expanded and positioned in such a way as to protect several arterial branches simultaneously, such as some or all of the major branches coming off of the aorta.

In various embodiments, the mesh material may have a pore size that is sized to allow vessel perfusion, while still preventing potential emboli from passing into an arterial branch. For example, in some embodiments, the mesh material may have a pore size of about 80-100 microns. In some embodiments, when expanded, the mesh material may have a concave surface and a convex surface. In various embodiments, this curvature may allow the convex surface to fit closely against the arterial wall and across the ostia of a plurality of arterial branches, such as two, three, four, five, or even more arterial branches. Thus positioned, the embolism protection device may collect on its arterial lumen-facing concave surface any potential emboli that may otherwise pass into an arterial branch.

In various embodiments, the embolism protection device may have an elongated shape, with a length of about 80-100 mm or even longer, such as 80 mm, 85 mm, 90 mm, 95 mm, 100 mm, or even longer, and an expanded width of about 10-20 mm, such as 10 mm, 12 mm, 14 mm, 16 mm, 18 mm, or 20 mm, to ensure that the device is of sufficient size to cover all of the major arterial branches coming off of the aorta, if desired. For example, in some embodiments, the embolism protection device may be positioned with a first end distal to the vertebral artery or even the axillary artery, and a second end extending proximally of the vertebral artery. When positioned in this manner, the device may protect the entire circulation from emboli arising near the aortic valve, including both the anterior circulation (e.g., the carotid arteries) and the posterior circulation (e.g., the vertebral arteries). One of skill in the art will appreciate that the dimensions of the embolism protection device may be tailored to suit a particular procedure or subject. For instance, an infant, child, or small adult may require a shorter and/or narrower device, whereas a larger adult may require a longer and/or wider device in order to properly span the selected arterial branches.

In use, in various embodiments, the embolism protection device may be advanced over a guidewire such as a TAD™ guidewire (Covidien, Mansfield Mass.), or another wire having a diameter of about 0.01-0.04 inches, such as about 0.014-0.035 inches. In some embodiments, the guidewire may have a tapered tip. Some embodiments of the embolism protection device may be used in conjunction with a concentric wire catheter guidewire system, such as those disclosed in U.S. Pat. No. 7,402,141.

In use, the embolism protection device may be guided along the guidewire with a support catheter such as a Judkins™ catheter, a Jackey catheter™, an internal mammary catheter, a Monorail™ catheter, or a Simmons™ type catheter. In various embodiments, once the device has been delivered to the desired location, the support catheter may be withdrawn, allowing the device to self-expand, thus protecting a plurality of arterial branches. Once the cardiac or vascular procedure has been completed, the support catheter may be advanced over the embolism protection device, collapsing it and permitting it to be withdrawn from the body. In some embodiments, prior to collapsing the device, a user may advance an evacuation catheter through the support catheter, and any debris collected in the embolism protection device may be suctioned from the device.

Additionally, although not required, in some embodiments, the device may include one or more stabilization features adapted to retain the device in a desired location within the arterial lumen. For instance, one or more wire stabilization loop features may be included proximal and/or distal of the embolism protection device that may be sized and positioned to exert gentle pressure against the arterial walls, thus stabilizing the position of the embolism protection device. Although wire stabilization loops are illustrated herein, one of skill in the art will appreciate that any other stabilization feature may be substituted. In some embodiments, no stabilization features are employed.

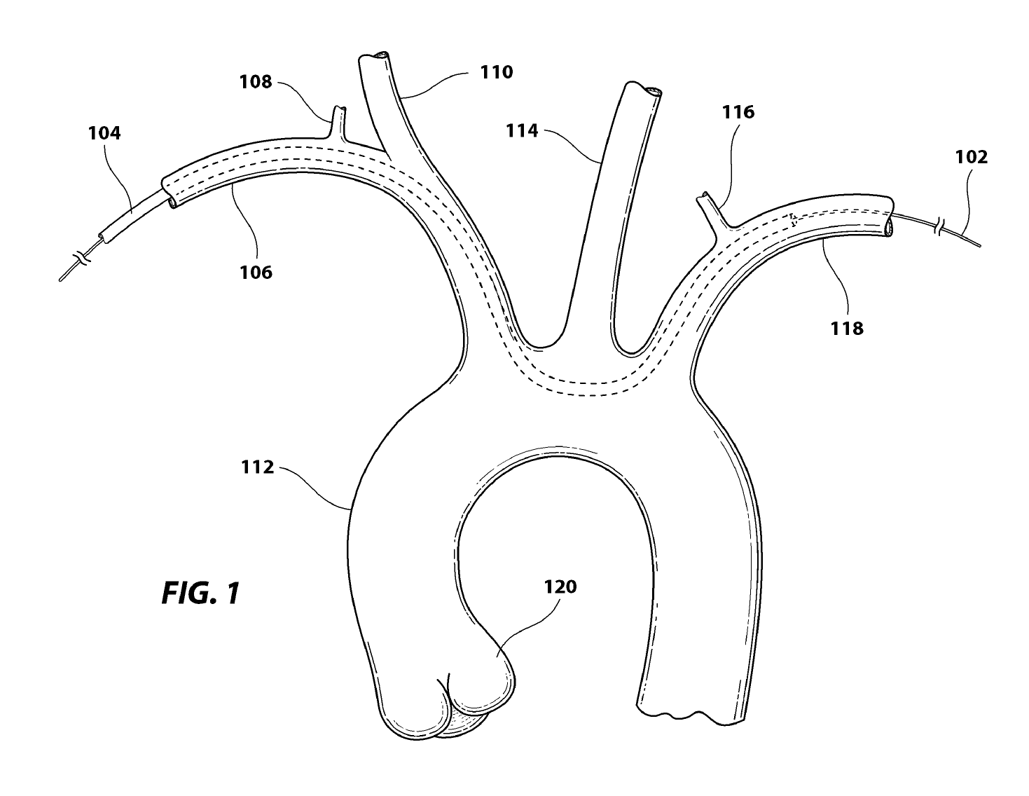

As shown in FIG. 1, a guidewire 102 and a support catheter 104 may be used to deliver the embolism protection device disclosed herein to a desired location within an artery, such as the subclavian artery 106, aorta 112, and/or right brachiocephalic artery 118, all adjacent the aortic valve 120. In this position, the embolism protection device may protect the right vertebral artery 108, the innominate artery 110, the left common carotid artery 114, the left vertebral artery 116, or a combination thereof. In various embodiments, guidewire 102 may be a TAD™ guidewire (Covidien, Mansfield Mass.), or another wire having a diameter of about 0.01-0.04 inches, such as about 0.014-0.035 inches. In some embodiments, the guidewire may have a tapered tip, and in some embodiments, a concentric wire catheter guidewire system may be used, such as those disclosed in U.S. Pat. No. 7,402,141.

In various embodiments, the length of guidewire 102 may be between about 100 cm and about 500 cm, such as about 150 cm-300 cm, but other sizes may be substituted for particular subjects, procedures, or access routes. In various embodiments, once guidewire 102 has been positioned as desired, the embolism protection device (not shown) may be advanced along guidewire 102 in a collapsed state inside of support catheter 104, which may be a Judkins™ catheter, a Jackey catheter™, an internal mammary catheter, a Monorail™ catheter, or a Simmons™ type catheter in accordance with various embodiments.

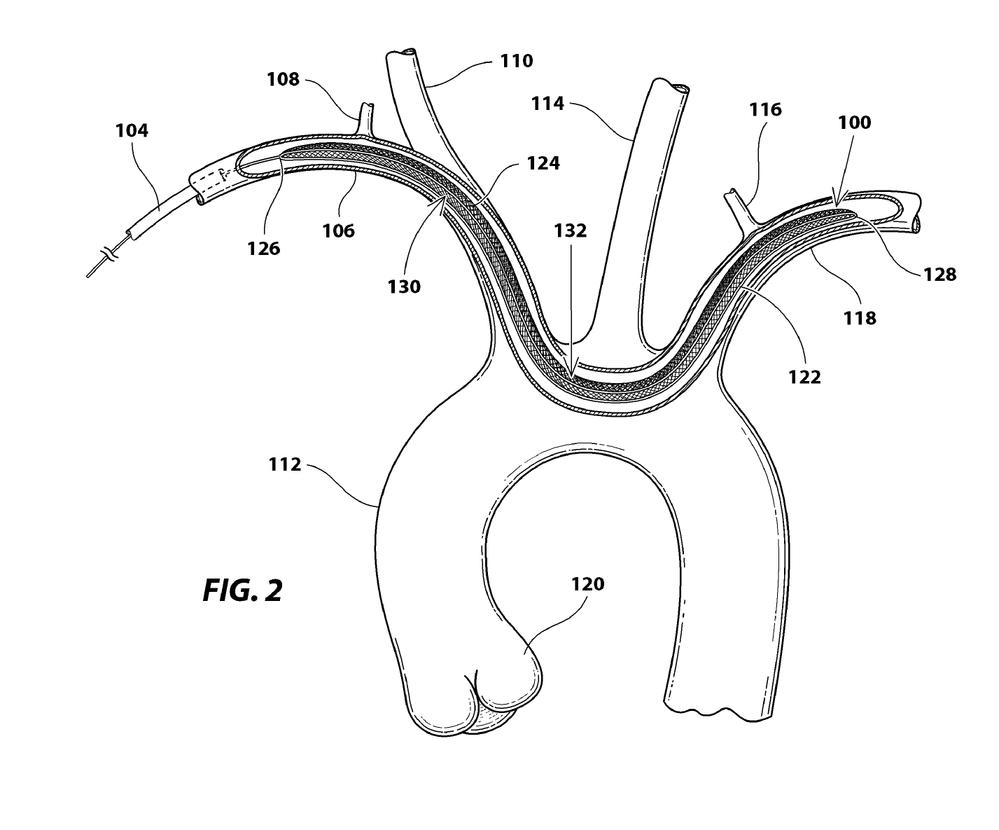

FIG. 2 illustrates an example of an embolism protection device 100 after support catheter 104 (which also may be referred to herein as a sheath) has been retracted to expose embolism prevention device 100. In various embodiments, when embolism protection device 100 is unsheathed, the compliant, elongated frame 122 and mesh material 124 may self-expand, forming a screen over the ostia of a plurality of arterial branches. In particular embodiments, embolism protection device 100 may be positioned with a proximal end 126 in subclavian artery 106 and a distal end 128 in the right brachiocephalic artery 118, distal to the left vertebral artery 116. In this position, embolism protection device 100 may protect the right vertebral artery 108, the innominate artery 110, the left common carotid artery 114, and the left vertebral artery 116, from emboli originating near aortic valve 120.

In various embodiments, both compliant elongated frame 122 and mesh material 124 may both be formed from a material having a shape memory function, such as Nitinol™. In various embodiments, mesh material 124 may have a pore size that is sized to allow red blood cells and other blood components to pass freely therethrough, while still preventing potential emboli from passing into an arterial branch. For example, in some embodiments, mesh material 124 may have a pore size of from about 80 microns to about 100 microns, or from about 90 microns to about 100 microns.

In some embodiments, when expanded, mesh material 124 may have a concave surface 130 and a convex surface 132. In various embodiments, this curvature may allow the convex surface 130 to fit closely against the arterial wall and across the ostia of a plurality of arterial branches, such as two, three, four, five, or even more arterial branches. Thus positioned, embolism protection device 100 may collect on its arterial lumen-facing concave surface 130 any potential emboli that may otherwise pass into an arterial branch.

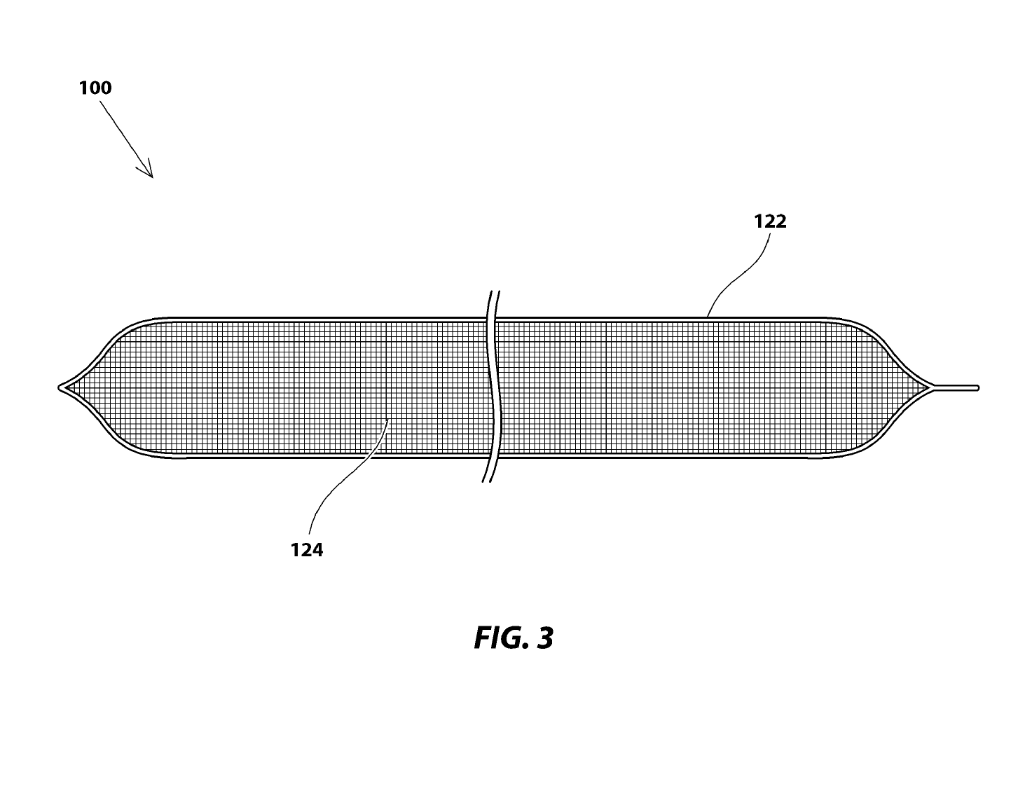

FIG. 3 shows a close-up view of the embolism protection device of FIGS. 1 and 2 in an expanded state. In various embodiments, embolism protection device 100 may have an elongated shape, and compliant elongated frame 122 and mesh material 124 may have a length of about 80-100 mm or even longer, and an expanded width of about 10-20 mm to ensure that the device is of sufficient size to cover all of the major arterial branches coming off of the aorta.

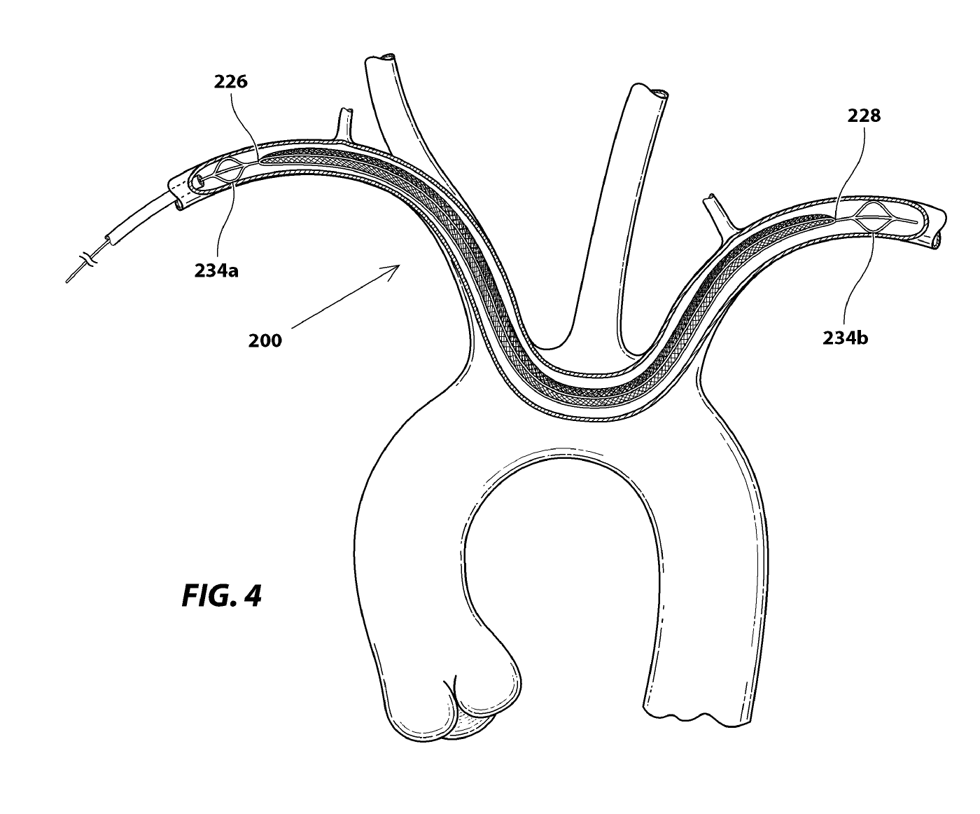

Turning now to FIG. 4, optionally in some embodiments, embolism protection device 200 may include one or more stabilization features adapted to retain the device in a desired location within the arterial lumen. For instance, one or more wire stabilization loop features 234a, 234b may be included adjacent a proximal end 226 and/or distal end 228 of embolism protection device 200. In various embodiments, such wire stabilization loops 234a, 234b may be sized and positioned to exert gentle pressure against the arterial walls, thus stabilizing the position of embolism protection device 200. Although wire stabilization loops are illustrated herein, one of skill in the art will appreciate that any other stabilization feature may be substituted. In various embodiments, no stabilization features are employed.

In one specific, non-limiting example, an embolism protection device in accordance with the present disclosure may be used to protect the brain of a subject undergoing a TAVI procedure or other percutaneous valve procedure, a coronary bypass surgery, or a heart valve surgery. In this example, a guidewire may be advanced through the vasculature using a right radial artery/right brachial artery approach, passing through the left subclavian artery and terminating in the right brachiocephalic artery, for instance distal to the left vertebral artery. An embolism protection device may then be advanced in a collapsed state within a support catheter until the distal end of the embolism protection device is in the right brachiocephalic artery, preferably distal to the left vertebral artery. The support catheter may then be withdrawn, allowing the elongated, compliant frame and mesh material of the embolism protection device to self-expand, covering the ostia of some or all of the major branches arising from the aorta.

A TAVI or other procedure (such as a coronary bypass surgery or a heart valve surgery) may then be performed, and the concave front surface of the embolism protection device may capture potential emboli, while allowing blood to flow freely through the mesh material. Once the procedure has been completed, the support catheter may be advanced over the embolism protection device, causing it to collapse inside the lumen of the catheter. In this collapsed state, the embolism protection device and support catheter may be withdrawn, along with or followed by the guidewire.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An intravascular embolism protection device comprising:
an elongated, compliant frame comprising a material having a shape-memory function;
a mesh material coupled to the elongated compliant frame, the mesh material having a pore size selected to allow blood to pass therethrough while retaining potential emboli;
wherein the embolism protection device has a collapsed state wherein the embolism protection device is configured to fit within the lumen of a support catheter and an expanded state wherein the embolism protection device is configured to cover a plurality of arterial branch ostias.

2. The intravascular embolism protection device of claim 1, wherein the elongated, compliant frame has a length dimension of about 80-100 mm.

3. The intravascular embolism protection device of claim 1, wherein the elongated, compliant frame has a width dimension of about 10-20 mm when in the expanded state.

4. The intravascular embolism protection device of claim 1, wherein the mesh material has a pore size of about 80-100 mm.

5. The intravascular embolism protection device of claim 1, wherein the mesh material has a pore size of about 100 mm.

6. The intravascular embolism protection device of claim 1, wherein the mesh material has a convex surface adapted to fit closely against an arterial wall and a concave surface adapted to capture potential emboli.

7. The intravascular embolism protection device of claim 1, wherein the embolism protection device is sized to cover the ostia of at least two arterial branches arising from the aorta.

8. The intravascular embolism protection device of claim 1, wherein the embolism protection device is sized to cover the ostia of at least three arterial branches arising from the aorta.

9. The intravascular embolism protection device of claim 1, wherein the embolism protection device is sized to cover the ostia of at least four arterial branches arising from the aorta.

10. The intravascular embolism protection device of claim 1, wherein the embolism protection device is sized to span a distance between a left subclavian artery and a right brachiocephalic artery.

11. The intravascular embolism protection device of claim 10, wherein the embolism protection device is sized to span a distance between a proximal position in the left subclavian artery proximal to a right vertebral artery and a distal position in the right brachiocephalic artery distal to a left vertebral artery.

12. The intravascular embolism protection device of claim 1, wherein the embolism protection device further comprises at least one stabilization feature.

13. The intravascular embolism protection device of claim 1, wherein the at least one stabilization feature comprises a wire stabilization loop.

14. The intravascular embolism protection device of claim 1, wherein the embolism protection device comprises a first wire stabilization loop distal to a distal end of the elongated compliant frame and a second stabilization loop proximal to a proximal end of the elongated compliant frame.

15. A method of protecting a subject from embolism during a percutaneous valve procedure, comprising:
advancing a guidewire through the vasculature of the subject through a left subclavian artery and to a right brachiocephalic artery;
advancing an embolism protection device over the guidewire to the right brachiocephalic artery, wherein the embolism protection device is in a collapsed state within a support catheter;
withdrawing the support catheter and allowing the embolism device to self-expand to cover the ostia of two or more arterial branches arising from a subclavian artery, an aorta, and/or a right brachiocephalic artery;
performing the percutaneous valve procedure;
advancing the support catheter to collapse the embolism protection device; and
removing the embolism protection device, support catheter, and guidewire from the vasculature of the subject.

16. The method of claim 15, wherein the embolism protection device is sized and positioned to cover the ostia of at least three arterial branches arising from the subclavian artery, the aorta, and/or the right brachiocephalic artery.

17. The method of claim 15, wherein the embolism protection device is sized and positioned to cover the ostia of at least four arterial branches arising from the subclavian artery, the aorta, and/or the right brachiocephalic artery.

18. The method of claim 15, wherein the at least two arterial branches comprise at least two of a left vertebral artery, an innominate artery, a left common carotid artery and a left vertebral artery.

19. The method of claim 15, wherein the at least two arterial branches comprise at least three of a left vertebral artery, an innominate artery, a left common carotid artery and a left vertebral artery.

20. The method of claim 15, wherein the at least two arterial branches comprise at a left vertebral artery, an innominate artery, a left common carotid artery and a left vertebral artery.
